(19) United States
(12) Patent Application Publication
(10) Pub. No.: US 2026/0131066 A1
(43) Pub. Date: May 14, 2026

(54) MEDICAL DEVICES AND RELATED SYSTEMS AND METHODS

(71) Applicants: Boston Scientific Scimed, Inc., Maple Grove, MN (US); BOSTON SCIENTIFIC MEDICAL DEVICE LIMITED, Galway (IE)

(72) Inventors: Nitesh Ghananil BAVISKAR, Pune (IN); Benjamin CLEVELAND, Bellingham, MA (US); Snehal PATIL, Pune (IN); Shrikant Vasant RAUT, Pune (IN); John SUBASIC, Sutton, MA (US)

(73) Assignees: Boston Scientific Scimed, Inc., Maple Grove, MN (US); BOSTON SCIENTIFIC MEDICAL DEVICE LIMITED, Galway (IE)

(21) Appl. No.: 19/386,608

(22) Filed: Nov. 12, 2025

Related U.S. Application Data
(60) Provisional application No. 63/720,419, filed on Nov. 14, 2024.

(51) Int. Cl. A61M 1/00; A61B 1/015

(52) U.S. Cl. CPC A61M 1/7415; A61B 1/015

(57) ABSTRACT

Medical devices and related systems and methods are described. The medical device may include a handle and a sheath extending from the handle, wherein the handle includes a hub. The hub may define a proximal opening and a chamber. The sheath may define a lumen in fluid communication with the proximal opening and the chamber. The hub may include at least one arm extending radially outward from the hub. An end of the at least one arm may be curved and may include a wheel or a concave support.

### CROSS-REFERENCE TO RELATED APPLICATION(S)

This application claims the benefit of priority to U.S. Provisional Application No. 63/720,419, filed on Nov. 14, 2024, which is incorporated by reference herein in its entirety.

### TECHNICAL FIELD

Aspects of the present disclosure generally relate to medical devices and medical systems. In particular, some aspects relate to medical devices and systems having a handle to control suction and movement of a shaft.

### BACKGROUND

Medical devices such as scopes and sheaths are often inserted into the body of a patient to perform a therapeutic and/or diagnostic procedure inside the body. Various features of the scope and/or sheath may assist in performing a therapeutic and/or diagnostic procedure inside the subject's body, e.g., including supply of irrigation or suction. Controlling distal and proximal movement of a shaft of the scope and controlling suction via a sheath surrounding the scope may require multiple hands and maintaining an uncomfortable position of the hands, and/or may require multiple operators. Such awkward positioning can lead to fatigue over time and potentially increase risk for the patient.

### SUMMARY

Each of the aspects disclosed herein may include one or more features described in connection with any of the other disclosed aspects.

The present disclosure includes medical devices and related systems and methods. For example, the present disclosure includes a medical device comprising a handle including a hub and a sheath extending from the handle. The hub of the handle may define a proximal opening and a chamber. The sheath may define a lumen in fluid communication with the proximal opening and the chamber of the hub. Further, for example, the hub may include at least one arm extending radially outward from the hub. An end of the at least one arm may be curved and may include a wheel or a concave support.

According to some aspects of the present disclosure, the at least one arm may be integral with the hub. The handle may include a suction port comprising a vent in communication with the chamber and the lumen. The vent may include an aperture (e.g., within a wall of the suction port) and an actuator configured to cover and uncover the actuator. The at least one arm may be a single arm that includes a concave support proximate the proximal opening.

In some examples, the at least one arm may include a first arm and a second arm opposite each other. The end of each of the first arm and the second arm may include a wheel. At least one of the first arm or the second arm may be configured to flex radially inward relative to a central longitudinal axis of the hub.

In some examples, the handle may include a grippable portion extending along an axis parallel to a central longitudinal axis of the hub. For example, an outer surface of the grippable portion may include a plurality of protrusions. In some examples, the handle may include a grippable portion extending along an axis transverse to a central longitudinal axis of the hub.

In some examples, the at least one arm may be a single arm. The end of the single arm may be distal to a proximal-most end of the hub. The hub may include an aperture and the single arm may include a wheel aligned with the aperture. For example, the single arm may be configured to flex radially inward toward the aperture.

In some examples, the handle may include a seal around the proximal opening. The hub may include an inner opening distal to the proximal opening. The inner opening may include a seal. The handle may include a finger rest. For example, the hub, the suction port, or both may include a finger rest.

The present disclosure also includes a medical device comprising a handle including a hub and a suction port. The hub may define a proximal opening and a chamber. The medical device may comprise a sheath extending from the handle. The sheath may define a lumen in fluid communication with the chamber and the suction port. The hub may include an arm extending radially outward from the hub. An end of the arm may include a wheel and may be distal to a proximal-most end of the hub.

According to some aspects of the present disclosure, the hub may include an aperture. The arm may be configured to flex radially inward such that at least a portion of the wheel extends through the aperture. The arm may be positioned opposite to the suction port relative to a central longitudinal axis of the hub.

The present disclosure further includes a medical device comprising a handle and a sheath extending from the handle. The hub may define a proximal opening and a chamber. The sheath may define a lumen in fluid communication with the chamber and the suction port. The hub may include an arm extending radially outward from the hub. An end of the arm may be proximal to a proximal-most end of the hub. The end may be curved and may include a concave support. In some examples, the suction port may include a grippable portion comprising a plurality of protrusions.

### BRIEF DESCRIPTION OF THE FIGURES

The accompanying drawings, which are incorporated in and constitute a part of this application, illustrate exemplary aspects that, together with the written descriptions, serve to explain the principles of this disclosure. Each figure depicts one or more exemplary aspects according to this disclosure, as follows:

FIG. 1 depicts an exemplary medical system that includes a medical device configured to receive a medical instrument such as a scope, according to some aspects of the present disclosure.

FIG. 2 depicts a perspective view of the medical device of FIG. 1.

FIGS. 3A and 3B depict another exemplary medical device, according to some aspects of the present disclosure.

FIGS. 4A and 4B depict another exemplary medical device, according to some aspects of the present disclosure.

### DETAILED DESCRIPTION

Particular aspects of the present disclosure are described in greater detail below. The terms and definitions provided herein control, if in conflict with terms and/or definitions incorporated by reference. The term “distal” refers to a portion farthest away from a user when introducing a device into a subject (e.g., patient). By contrast, the term “proximal” refers to a portion closest to the user when placing the device into the subject. Proximal and distal directions are labeled with arrows marked “P” and “D,” respectively, throughout various figures.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range. As used herein, the phrase “based on” is understood to be equivalent to the phrase “based at least on,” unless indicated otherwise. The term “or” is used disjunctively, such that “at least one of A or B” includes, (A), (B), (A and A), (A and B), etc.

Although scopes and ureteroscopes are referenced herein for illustration purposes, it will be appreciated that the disclosure encompasses any suitable medical device configured to allow a user to access and view internal body anatomy of a subject and/or to deliver medical instruments, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the features described herein.

The medical devices and related systems described in this disclosure may include a handle configured to be operated with only one hand of a user, e.g., a non-dominant hand of the user. The handle of the medical device may include a hub. The medical device also may include a sheath extending distally from the handle, e.g., the sheath defining a lumen in fluid communication with a proximal opening of the hub. For example, the proximal opening may be configured to receive a medical instrument such as a scope. The hub may include features to support such a medical instrument and facilitate movement of the medical instrument relative to the medical device.

For example, the hub of the medical device may include one or more arms each having an end including one or more wheels or curved supports. The wheel(s) or curved support(s) may be positioned relative to the hub to support the medical instrument. In some aspects, the medical system includes the medical instrument, e.g., a scope having a handle and a shaft extending from a distal end of a handle. The scope may be operated by the other hand of the user, such as a dominant hand of the operator. The shaft of the scope may be extended through the medical device via the hub of the handle, such that the scope shaft extends through the sheath of the medical device. The user may use a digit (e.g., a thumb of their non-dominant hand) to distally extend and proximally retract the medical instrument shaft through the hub and the sheath of the medical device. The handle of the medical device may further include a suction port configured to apply negative pressure to the lumen of the sheath. The suction port may include a vent with an aperture that may be covered or uncovered to control pressure.

Reference will now be made in detail to examples to help illustrate aspects of the present disclosure through the accompanying drawings. Wherever possible, the same reference numbers will be used throughout the drawings to refer to the same or like parts.

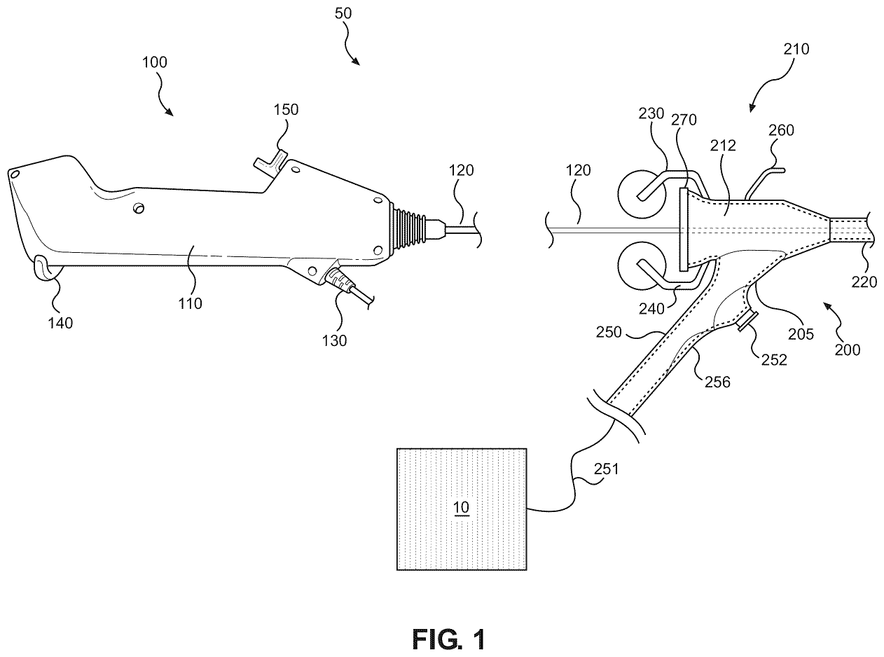

FIG. 1 depicts an exemplary medical system 50 according to aspects of the present disclosure. Medical system 50 includes a medical device 200 and a medical instrument such as a scope 100. For example, medical device 200 may be configured to receive a portion of a shaft 120 of scope 100. Scope 100 as shown includes a handle 110 coupled to shaft 120, a port 150, an actuator 140 that controls the shaft 120, and an umbilicus 130 to supply power and/or transmit data to electronic components of scope 100.

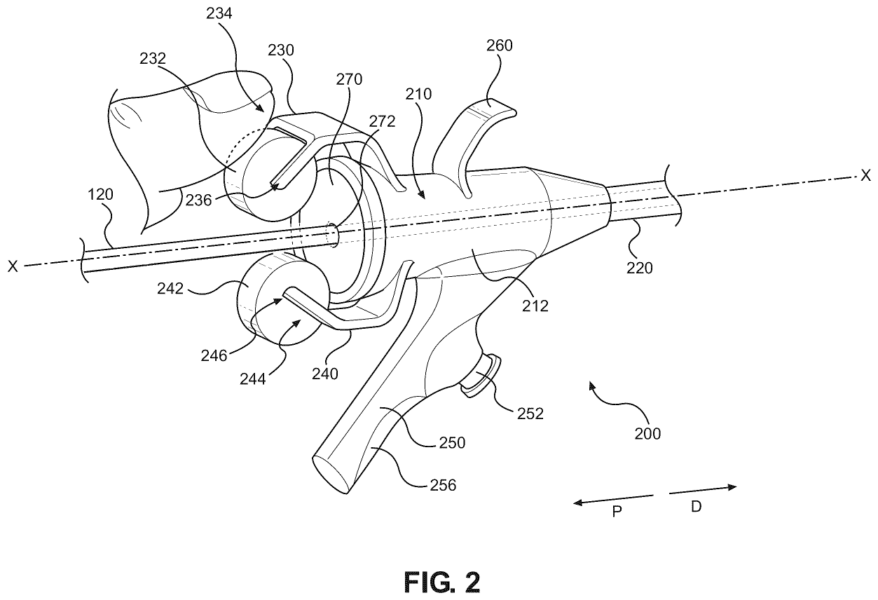

As illustrated in FIGS. 1 and 2, medical device 200 includes a handle 205 with a hub 210 and a suction port 250. Hub 210 may be generally cylindrical in shape as shown, however, this is exemplary. Optionally, hub 210 may be tapered such that the proximal end of hub 210 has a greater cross-sectional dimension that the distal end of hub 210. Handle 205 may be configured to be controlled with one hand of a user, e.g., during a medical procedure. For example, handle 205 may be controlled by a non-dominant hand of the user.

Hub 210 may include a chamber 212 in fluid communication with suction port 250 and with a lumen of sheath 220. Hub 210 may be configured to slidably receive at least a portion of shaft 120 extended therethrough such that shaft 120 may be slidable in the proximal and distal direction within chamber 212 and into sheath 220. Handle 205 may include a proximal opening 272 configured to receive shaft 120 extended therethrough, e.g., hub 210 including a seal 270 around proximal opening 272 to provide a fluid-tight seal with shaft 120.

Sheath 220 may be coupled to, or integral with, handle 205. As mentioned above, sheath 220 may define a lumen sized and shaped to receive a medical instrument (such as shaft 120 of scope 100) and to provide space between an inner surface of sheath 220 and an outer surface of the medical instrument for the application of suction through sheath 220.

In order to facilitate insertion and movement of a medical instrument into medical device 200, hub 210 may include one or more arms extending radially outward from hub 210. In this example, hub 210 includes a first arm 230 and a second arm 240. In other aspects, hub 210 only includes one single arm, e.g., first arm 230. Each of first arm 230 and second arm 240 may be integrally formed with hub 210 or fixedly coupled to hub 210. Arms 230, 240 may be at least partially flexible to allow radial movement towards and away from hub 210. For example, each arm 230, 240 may be coupled to hub 210 via a hinge or integrated into hub 210 via a living hinge. Arms 230, 240 may be curved. For example, each arm 230, 240 may extend radially outward from hub 210, curve proximally around the proximal end of hub 210 and radially inward toward a central longitudinal axis X of hub 210. Proximal ends of arms 230, 240 may be proximal to hub 210 (e.g., proximal to a proximal-most end of hub 210).

Arms 230, 240 may comprise a flexible and/or elastic material, such that arms 230, 240 can flex relative to hub 210 as mentioned above (e.g., via a living hinge) when pressed by the hand of a user or can flex relative to hub 210 along a length of respective arms 230, 240. The operator may apply a force (e.g., with one or more digits of the non-dominant hand) to respective arm 230, 240 to flex the respective arm 230, 240 radially inward toward axis X. Arms 230, 240 may be configured to return to a pre-flexed, default position after the operator stops applying force. For example, applying a radially-inward directed force at the proximal end of first arm 230 may cause first arm 230 to flex at the connected, distal end and the proximal end flexes toward the axis X. Ceasing application of force at the proximal end of first arm 230 may permit arm 230 to automatically transition to its pre-flexed, default position such that the proximal end flexes away from the axis X.

The proximal end of each arm 230, 240 may include a wheel 232, 242. The size of wheels 232, 242 may be the same or different from each other. For example, the diameter of wheel 232 coupled to first arm 230 may be greater than the diameter of wheel 242 coupled to second arm 240. Wheels 232, 242 may be coupled to arms 230, 240 via respective slots 234, 244. Slots 234, 244 may include one or more inwardly extending protrusions 236, 246 (e.g., pins), respectively, configured to be received within corresponding central holes of respective wheels 232, 242. Wheels 232, 242 may be rotatable about respective protrusions 236, 246.

Suction port 250 of handle 205 may be transverse to longitudinal axis X. Suction port 250 may be coupled to a vacuum source 10 in order to provide negative pressure through sheath 220 during a medical procedure. For example, suction port 250 may be fluidly connected to vacuum source 10 via tubing 251. Suction port 250 may include a vent 252 useful for controlling pressure. Vent 252 may include an aperture within the wall of suction port 250 and a mechanism, e.g., an actuator, to cover and uncover the aperture to increase or decrease suction pressure, respectively. Optionally, vent 252 may include a biasing element (e.g., a spring or other biasing element) that biases the actuator of vent 252 in either an open position where the aperture of vent 252 is uncovered, or a closed position where the aperture of vent 252 is covered. For example, a user may press the actuator (button, lever, slider, knob, etc.) of vent 252 over the aperture to overcome a biasing force and uncover the aperture, thereby decreasing suction pressure. When the aperture of vent 252 is uncovered, medical device 200 may provide less suction (e.g., lower volumetric flow rate of suction) through sheath 220 compared to when the aperture of vent 252 is covered.

Optionally, handle 205 may include surface features that are ergonomic or otherwise facilitate gripping by the user. For example, suction port 250 may include a grippable portion 256 that includes a groove, cut, or notch configured to receive one or more digits of the user's hand. Grippable portion 256 may extend along an axis transverse to central longitudinal axis X of hub 210. Additionally or alternatively, grippable portion 256 (e.g., an outer surface of grippable portion 256) may include one or more protrusions to increase friction when gripped by the hand of a user. As shown in this example, grippable portion 256 is along suction port 250 proximate vent 252, although other locations are possible, e.g., based on where the user holds handle 205. Additionally or alternatively to grippable portion 256, handle 205 may include a finger rest 260, e.g., on hub 210, to receive another finger, e.g., index or middle finger, of the user's hand. Finger rest 260 may extend radially outward from hub 210 relative to axis X. Finger rest 260 may be integral with hub 210 or coupled to hub 210. Finger rest 260 may include a curved surface. Finger rest 260 and/or grippable portion 256 may facilitate an ergonomic hand grip position, e.g., to reduce hand and finger fatigue of the operator.

In an exemplary procedure utilizing medical device 200 to treat a subject, handle 205 of medical device 200 may be held and operated by the non-dominant hand of the user. The dominant hand of the user may be used to control scope 100. Sheath 220 of medical device 200 may be introduced into a passageway or an organ of the subject's body so that the distal end of sheath 220 is proximate a target site (e.g., within the urinary or gastrointestinal tract). The user may introduce shaft 120 of scope 100 into medical device 200 through proximal opening 272 and advance shaft 120 through the lumen of sheath 220. The non-dominant hand of the user may adjust the position of shaft 120 relative to sheath 220 by pressing wheel 232 such that first arm 230 flexes radially inward to contact shaft 120. Shaft 120, in turn, may contact wheel 242 of second arm 240 such that shaft 120 is between wheels 232, 242. The user may rotate wheel 232 clockwise or counterclockwise to control proximal and distal movement of shaft 120 relative to sheath 220. The opposite wheel 242 may rotate in the opposite direction as wheel 232.

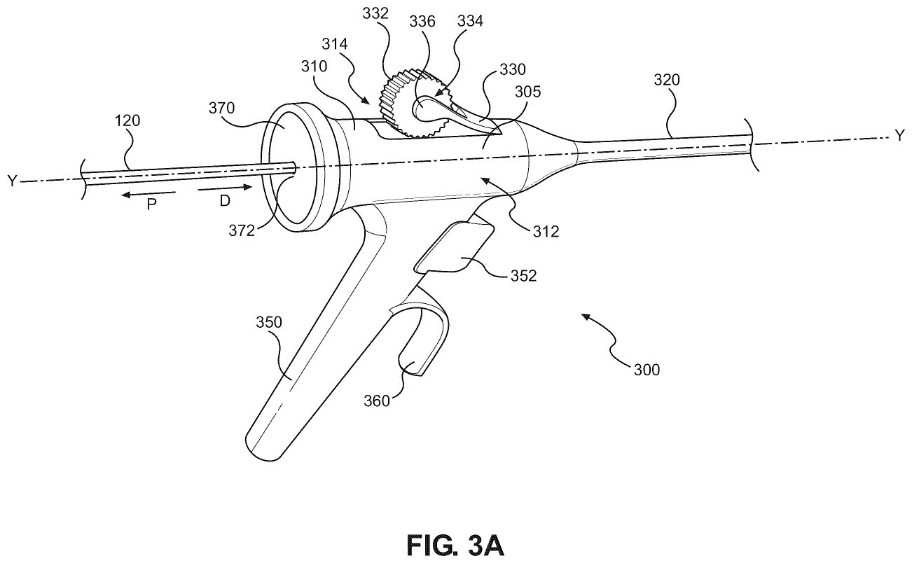

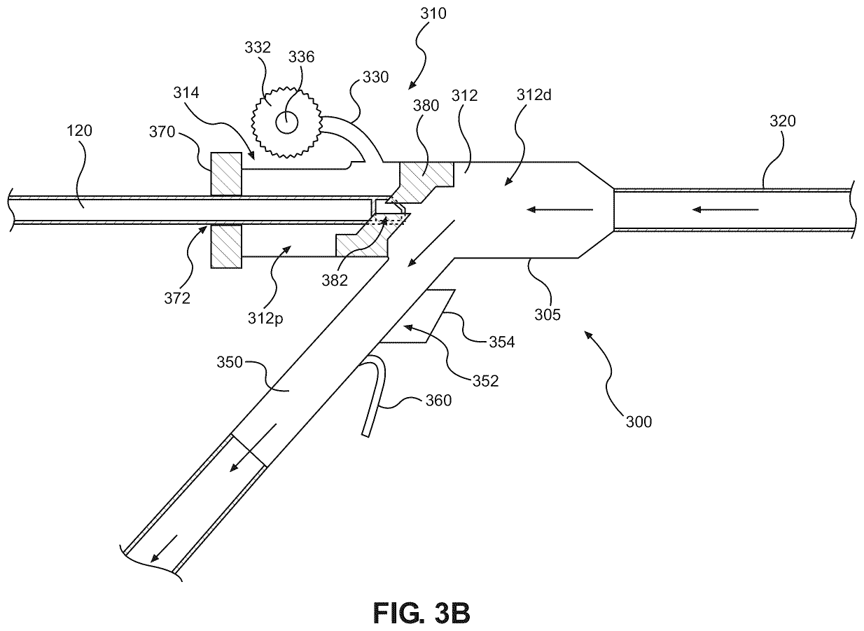

FIGS. 3A-3B depict another exemplary medical device 300, which may include any of the features of medical device 200 unless otherwise described. Medical device 300 includes a handle 305 and a sheath 320 that defines a lumen. Handle 305 may include a hub 310 and a suction port 350. Hub 310 defines a chamber 312 in fluid communication with a proximal opening 372 configured to receive a medical instrument, e.g., shaft 120 of scope 100, and in fluid communication with suction port 350 and the lumen of sheath 320.

As shown in FIG. 3B, handle 305 includes a first seal 370 proximate proximal opening 372. Handle 305 also includes an inner opening 382 within chamber 312 and a second seal 380 proximate inner opening 382. Second seal 380 is distal relative to first seal 370 and proximal to a junction of chamber 312 and suction port 350. For example, a first, proximal portion 312p of chamber 312 may be defined between seals 370, 380 and a second, distal portion 312d of chamber 312 may be defined between second seal 380 and the distal end of handle 305. Accordingly, suction port 350 may be in fluid communication with distal portion 312d of chamber 312 and the lumen of sheath 320, but can be fluidly isolated from proximal portion 312p of chamber 312 when second seal 380 is closed (e.g., via a fluid-tight seal around a shaft of a medical instrument, e.g., shaft 120 of scope 100). Similarly, proximal portion 312p of chamber 312 may be fluidly isolated from the lumen of sheath 320 when second seal 380 is closed. Therefore, proximal portion 312p of chamber 312 need not be isolated from the external environment in order to provide and control negative pressure via suction port 350. According to some aspects of the present disclosure, seal 370 and/or seal 380 may form a fluid-tight seal when a shaft of a medical instrument (e.g., shaft 120 of scope 100) is received within respective openings 372, 382, or when the shaft of the medical instrument is omitted (e.g., not received).

Hub 310 may include an arm 330. Arm 330 may have any of the features of first and/or second arm 230, 240 discussed above. For example, arm 330 may extend radially outward from hub 310 to an end, e.g., a curved proximal end that includes a wheel 332. Arm 330 may include a curve along a length of arm 330 such that arm 330 curves radially inward. Wheel 332 may be coupled to arm 330 via a slot 334 along arm 330. One of slot 334 or wheel 332 may include a protrusion or pin 336 and the other of the slot 334 or wheel 332 may include a corresponding hole for receiving pin 336. Wheel 332 may be rotatable about pin 336. According to aspects of the present disclosure, hub 310 may include one single arm 330, or may include one or more arms having any features of arms 230, 240 (e.g., in addition to arm 330).

Wheel may be aligned with an aperture 314 of hub 310. For example, aperture 314 may be defined through the wall of hub 310. Aperture 314 may be in fluid communication with proximal portion 312p of chamber 312. Arm 330 may be configured to flex radially inward such that at least a portion of wheel 332 extends through aperture 314. For example, arm 330 may be integral with hub 310 (e.g., via a living hinge) or may be coupled with a connection to hub 310 that permits arm 330 to flex or bend at the connected end of arm 330.

When a medical instrument is extended through sheath 320, e.g., shaft 120 enters proximal opening 372 and extends through proximal portion 312p of chamber 312, arm 330 may be flexed radially inward such that wheel 332 contacts shaft 120. While wheel 332 is flexed to contact shaft 120, rotating wheel 332 in a first direction (e.g., clockwise) may distally extend shaft 120 relative to sheath 320 and rotating wheel 332 in a second, opposite direction (e.g., counterclockwise) may proximally retract shaft 120 relative to sheath 320.

With continued reference to FIGS. 3A-3B, suction port 350 may include a vent 352 having any of the features of vent 252 discussed above to control suction pressure in use. In an exemplary medical procedure using medical device 300, a digit (e.g., an index finger) of the user's hand may be used to control the actuator of vent 352.

Optionally, handle 305 may include a finger rest. For example, suction port 350 may include a finger rest 360 having any of the features of finger rest 260 of medical device 200. In this example, finger rest 360 extends from suction port 350. Finger rest 360 may be integral with, or coupled to, suction port 350. Space between finger rest 360 and suction port 350 may be configured to receive one of more of a middle finger, ring finger, or pinky finger of the user's hand while an index finger of the user's hand is used to control vent 352.

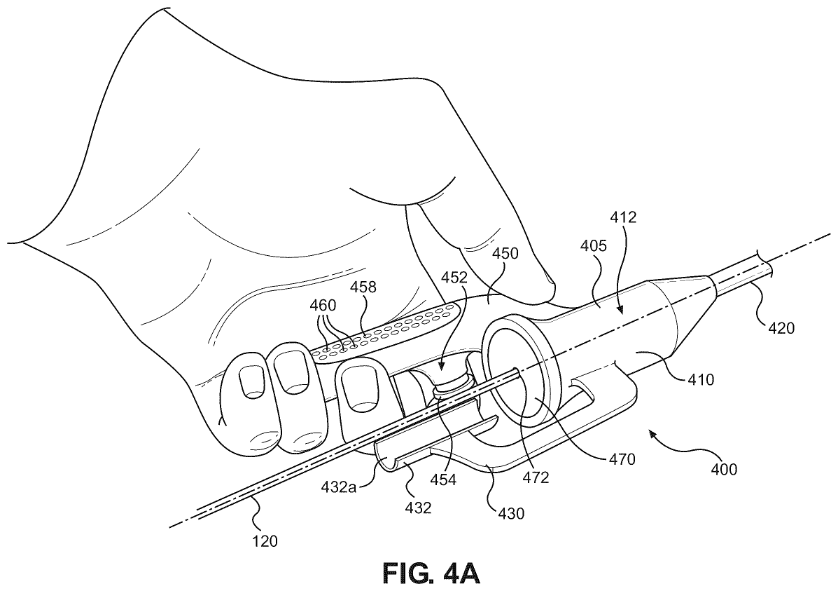

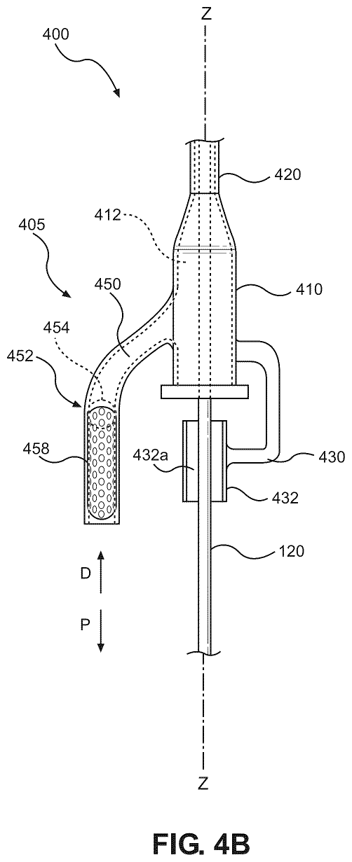

FIGS. 4A-4B depict another exemplary medical device 400 which may include any of the features of medical devices 200, 300 unless otherwise described. Medical device 400 includes a handle 405 and a sheath 420. Handle 405 may include a hub 410 defining a proximal opening 372 and a chamber 412, and a suction port 450. Sheath 420 extends distally from handle 405.

Hub 410 includes an arm 430, which may have any of the features of arms 230, 240, 330 discussed above. For example, arm 430 may be integral with or coupled to hub 410, and may extend proximally relative to hub, wherein arm 430 curves radially inward toward. According to aspects of the present disclosure, hub 410 may include a single arm 430, or may include one or more arms having any features of arms 230, 240, 330 (e.g., in addition to arm 430). At the proximal end, arm 430 include a concave support 432. That is, support 432 may include a concave surface 432a, such as a half-cylinder shape. The concave surface 432a at the end of arm 430 may be proximate proximal opening 472, e.g., in order to contact and support a medical instrument (e.g., shaft 120 of scope 100) inserted into proximal opening 472. For example, FIGS. 4A-4B shows shaft 120 aligned with the concave surface 432a of concave support 432. Shaft 120 may be slidably received within concave support 432 and slidable along concave surface 432a in the proximal and/or distal direction.

Suction port 450 may include a vent 452, which may include any of the features of vents 252, 352 discussed above to control suction pressure of medical device 400. In this example, suction port 450 includes a grippable portion 458 with surface features configured to facilitate a user's grip in use. Grippable portion 458 may be positioned opposite to vent 452 along suction port 450. Grippable portion 458 may include one or more protrusions, e.g., a plurality of protrusions 460, configured to increase friction with a digit or palm of a user when the user's hand grips handle 405. Plurality of protrusions 460 may help to prevent slippage between grippable portion 458 and the user's hand.

As shown in FIG. 4A, handle 405 may be configured to receive a hand of the operator (e.g., a non-dominant hand) and facilitate an ergonomic hand grip position. For example, suction port 450 may extend along an axis parallel to a central longitudinal axis of hub 410. Similarly, grippable portion 458 may extend along the axis parallel to the central longitudinal axis of hub 410. According to some aspects, suction port and arm 430 may be coplanar or approximately coplanar. In the example shown in FIG. 4A, a user's hand is depicted gripping handle 405 such that one or more digits and/or the palm contact grippable portion 458. While gripping suction port 450 of handle 405, another digit, e.g., the index finger, may be used to control vent 452. Further, one or more digits of the same hand may be used to slide shaft 120 relative to concave support 432. For example, an index finger may be used to control vent 452 and a thumb may be used to slide shaft 120 relative to concave support 432.

While principles of this disclosure are described herein with reference to illustrative examples for particular uses, it should be understood that the disclosure is not limited thereto. Those having ordinary skill in the art and access to the teachings provided herein will recognize additional modifications, uses, examples, and substitution of equivalents all fall within the scope of this disclosure.

What is claimed is:

1. A medical device comprising:
a handle including a hub that defines a proximal opening and a chamber; and
a sheath extending from the handle, the sheath defining a lumen in fluid communication with the proximal opening and the chamber,
wherein the hub includes at least one arm extending radially outward from the hub, and wherein an end of the at least one arm is curved and includes a wheel or a concave support.

2. The medical device of claim 1, wherein the at least one arm includes a first arm and a second arm opposite each other, the end of each of the first arm and the second arm including a wheel.

3. The medical device of claim 2, wherein at least one of the first arm or the second arm is configured to flex radially inward relative to a central longitudinal axis of the hub.

4. The medical device of claim 1, wherein the at least one arm is integral with the hub.

5. The medical device of claim 1, wherein the handle includes a grippable portion extending along an axis parallel to a central longitudinal axis of the hub.

6. The medical device of claim 5, wherein an outer surface of the grippable portion includes a plurality of protrusions.

7. The medical device of claim 1, wherein the handle includes a grippable portion extending along an axis transverse to a central longitudinal axis of the hub.

8. The medical device of claim 7, wherein the handle includes a finger rest.

9. The medical device of claim 1, wherein the at least one arm is a single arm, and wherein the end of the single arm is distal to a proximal-most end of the hub.

10. The medical device of claim 9, wherein the hub includes an aperture, and wherein the single arm includes a wheel aligned with the aperture.

11. The medical device of claim 10, wherein the single arm is configured to flex radially inward toward the aperture.

12. The medical device of claim 1, wherein the handle includes a suction port, the suction port comprising a vent in communication with the chamber and the lumen, and wherein the vent includes an aperture within a wall of the suction port and an actuator configured to cover and uncover the aperture of the vent.

13. The medical device of claim 1, wherein the handle includes a seal around the proximal opening.

14. The medical device of claim 13, wherein the hub includes an inner opening distal to the proximal opening, the inner opening including a seal.

15. The medical device of claim 1, wherein the at least one arm is a single arm that includes a concave support proximate the proximal opening.

16. A medical device comprising:
a handle including a hub and a suction port, wherein the hub defines a proximal opening and a chamber; and
a sheath extending from the handle, the sheath defining a lumen in fluid communication with the chamber and the suction port;
wherein the hub includes an arm extending radially outward from the hub, and wherein an end of the arm is distal to a proximal-most end of the hub, the end of the arm including a wheel.

17. The medical device of claim 16, wherein the hub includes an aperture and the arm is configured to flex radially inward such that at least a portion of the wheel extends through the aperture.

18. The medical device of claim 17, wherein the arm is positioned opposite to the suction port relative to a central longitudinal axis of the hub.

19. A medical device comprising:
a handle including a hub and a suction port, wherein the hub defines a proximal opening and a chamber;
a sheath extending from the handle, the sheath defining a lumen in fluid communication with the chamber and the suction port;
wherein the hub includes an arm extending radially outward from the hub, and wherein an end of the arm is proximal to a proximal-most end of the hub, the end being curved and including a concave support.

20. The medical device of claim 19, wherein the suction port includes a grippable portion comprising a plurality of protrusions.
